(19) United States
(12) Patent Application Publication
(10) Pub. No.: US 2015/0225748 A1
(43) Pub. Date: Aug. 13, 2015

(54) PROCESSES FOR ANAEROBIC BIOCONVERSION OF HYDROGEN-CONTAINING GASES TO OXYGENATED ORGANIC COMPOUNDS

(71) Applicants: Jianxin Du, Naperville, IL (US); Joshua A. Schumacher, Warrenville, IL (US); Andrew J. Heinen, Aurora, IL (US); Jian Xu, Naperville, IL (US)

(72) Inventors: Jianxin Du, Naperville, IL (US); Joshua A. Schumacher, Warrenville, IL (US); Andrew J. Heinen, Aurora, IL (US); Jian Xu, Naperville, IL (US)

(21) Appl. No.: 14/175,865

(22) Filed: Feb. 7, 2014

(51) Int. Cl. C12P 7/14; C12P 7/16

(52) U.S. Cl. CPC C12P 7/14; C12P 7/16

(57) ABSTRACT

Anaerobic processes for the bioconversion of syngas to oxygenated organic compounds in an aqueous menstruum are disclosed where exogenous carbon dioxide is used to provide a syngas-containing substrate gas having a desired electron to carbon ratio. The exogenous carbon dioxide contains free oxygen, and the aqueous menstruum withdrawn for product recovery is contacted with the exogenous carbon dioxide to reduce its oxygen concentration before being supplied as part of the substrate gas.

### FIELD OF THE INVENTION

This invention pertains to processes for anaerobically converting hydrogen-containing feed gas to oxygenated organic compounds, especially ethanol, propanol and butanol using an exogenous carbon dioxide-containing gas to enhance bioconversion of hydrogen.

### BACKGROUND

Anaerobic fermentations of hydrogen and carbon monoxide involve the contact of the substrate gas in an aqueous fermentation menstruum with microorganisms capable of generating oxygenated organic compounds. Particular interest has focused on the production of alcohols such as ethanol, propanol, i-butanol and n-butanol. The production of these alcohols requires significant amounts of hydrogen and carbon monoxide. For instance, the theoretical equations for the conversion of carbon monoxide and hydrogen to ethanol and acetic acid are:

6 CO+3 H₂O.C₂H₅OH+4 CO₂

6 H₂+2 CO₂.C₂H₅OH+3 H₂O

2 H₂ +2 CO.C₂H₃OOH

4 H₂+2 CO₂.C₂H₅OOH+2 H₂O.

The conversion of hydrogen involves the consumption of hydrogen and carbon dioxide. Where the feed gas contains carbon monoxide, carbon dioxide can be generated as is shown above. Carbon dioxide can be supplied with the feed gas. This H₂/CO₂ conversion, for purposes herein, it is referred to as the hydrogen conversion.

Gases containing one or both of carbon monoxide and hydrogen can be obtained from various sources. Typically the feed gas for carbon monoxide and hydrogen conversions is, or is derived from, a synthesis gas (syngas) from the gasification of carbonaceous materials, from partial oxidation or reforming of natural gas and/or biogas from anaerobic digestion or landfill gas or off-gas streams of various industrial methods such as off gas from coal coking and steel manufacture. These feed gases thus typically contain carbon monoxide, hydrogen, and carbon dioxide and usually contain other components such as water vapor, nitrogen, methane, ammonia, hydrogen sulfide and the like. (For purposes herein, all gas compositions are reported on a dry basis unless otherwise stated or clear from the context.)

Depending upon the source of the feed gas insufficient carbon dioxide and carbon monoxide, which generates carbon dioxide when anaerobically converted to alcohol, may be present to convert hydrogen. Accordingly, exogenous carbon dioxide may be supplied to the aqueous fermentation menstruum. In copending U.S. Provisional Patent Application Nos. 61/762,715 and 61/762,702, both filed Feb. 8, 2013, processes are disclosed for the anaerobic bioconversion of gas substrate comprising carbon monoxide, hydrogen and carbon dioxide in an aqueous menstruum in bioreactors characterized as having a substantially uniform aqueous menstruum composition and a substantially non-uniform substrate composition wherein the gas substrate has a ratio of electrons to carbon atoms in the range of about 5.2:1 to 6.8:1. In copending U.S. Provisional Patent Application No. 61/762,715, the use of carbon dioxide from fermentations of carbohydrates such as sugars and starches to produce alkanols is disclosed as a source of carbon dioxide for adjusting the electron to carbon ratio.

The microorganisms used for anaerobic fermentation of syngas are adversely affected by the presence of oxygen. Accordingly, any gases introduced into the aqueous fermentation menstruum should be substantially devoid of molecular oxygen. Carbon dioxide-containing gases emanating from the fermentation of carbohydrates typically contains a low concentration of oxygen. Carbon dioxide gas from other sources can also contain some oxygen. Thus, processes are desired for removing any oxygen contained in such carbon dioxide-containing gases prior to introduction into the aqueous fermentation menstruum, which processes are effective and require little capital and operating expense.

Typical processes for removing free oxygen from gases use chemical reagents to react with the oxygen such as bisulfite anion and reduction catalysts, both of which entail additional expense. Moreover, the chemical reagent used may not be advantageous for a fermentation process as it, or the reaction product generated during the oxygen removal unit operation, may adversely affect the microorganisms or recovery of the oxygenated organic compound.

### SUMMARY

In accordance with this invention processes, exogenous carbon dioxide is used as a part of the total substrate gas used to anaerobically bioconvert hydrogen-containing feed gas to oxygenated organic compounds to provide sought electron to carbon ratios in the substrate gas. The exogenously supplied carbon dioxide-containing gas contains oxygen which is efficiently removed by this process integration. By this invention it has been found that aqueous menstruum withdrawn from the fermentation unit operation has sufficient reductive potential to reduce the oxygen concentration in an exogenous carbon dioxide-containing gas. By using the aqueous menstruum, not only are costs to purchase and introduce chemical reagents to achieve oxygen removal avoided or reduced, but also, since the aqueous menstruum is used, concerns regarding adverse effects on the microorganisms and oxygenated organic compound product recovery are attenuated. In instances where the oxygen concentrations are so high that chemical reagents are required, the amounts required are significantly reduced.

In one broad aspect of the invention, continuous processes for the anaerobic bioconversion of a gas substrate comprising hydrogen and carbon dioxide, and optionally carbon monoxide, in an aqueous menstruum containing microorganisms suitable for converting said substrate to at least one oxygenated organic compound, comprise:
a. continuously introducing hydrogen-containing gas into a bioreactor assembly containing said aqueous menstruum as a portion of the gas substrate;
b. continuously introducing into said bioreactor assembly an exogenous carbon dioxide-containing gas, which introduction may be by one or more of direct introduction into said bioreactor assembly and admixture with at least a portion of the hydrogen-containing gas, said carbon dioxide-containing gas comprising a portion of the gas substrate;
c. maintaining contact between the aqueous menstruum and gas substrate under bioconversion conditions sufficient to bioconvert gas substrate to an oxygenated organic compound, said bioconversion conditions including an Effective Oxidation Reduction Potential of less than about −150, preferably less than about −250, millivolts, to provide an product-containing menstruum and a substrate depleted gas phase;
d. continuously withdrawing substrate depleted gas phase from said aqueous menstruum;
e. continuously or intermittently withdrawing a portion of said product-containing menstruum, said withdrawal being sufficient to maintain the oxygenated organic compound in said menstruum below a concentration that unduly adversely affects the microorganisms;
f. contacting at least a portion of the withdrawn product-containing menstruum with an exogenous, oxygen-containing, carbon dioxide gas under conditions sufficient to reduce the oxygen concentration of the carbon dioxide gas and to provide a spent menstruum;
g. passing at least a portion of the carbon dioxide gas having a reduced oxygen concentration of step (f) to the bioreactor assembly as at least a portion of the carbon dioxide-containing gas of step (b); and
h. recovering said oxygenated organic compound from the spent menstruum of step (f).

In the preferred processes of this invention, the exogenous carbon dioxide is used to provide a total substrate gas have an electron to carbon ratio of at least about 1:1; preferably in the range of about 2:1 to 6.8:1. Preferably the Effective Oxidation Reduction Potential of the aqueous medium contacting the exogenous, oxygen-containing carbon dioxide is less than about −150, preferably less than about −250, millivolts, and the oxygen concentration of the exogenous carbon dioxide-containing gas is reduced by at least 50%. In some instances, sufficient oxygen is removed from the carbon dioxide such that, based on the total volume of substrate gas introduced into the bioreactor assembly, the oxygen concentration is less than about 100 parts per million by volume. Preferred sources of exogenous carbon dioxide-containing gas are off gases from carbohydrate fermentations.

In preferred processes of this invention, at least a portion, often an aliquot portion, of the withdrawn substrate depleted gas phase of step (d) is admixed with the exogenous carbon dioxide-containing gas prior to contact with the withdrawn alcohol-containing menstruum. The depleted gas phase contains oxygen reducing moieties and thus adds to the reducing potential of the aqueous menstruum. Typically the pressure of the exogenous carbon dioxide-containing gas has to be increased for introduction into the withdrawn aqueous menstruum for removal of oxygen and into the bioreactor assembly, or hydrogen-containing gas that is to be introduced into the bioreactor assembly. Advantageously, the withdrawn substrate depleted gas is admixed with the exogenous carbon dioxide-containing gas, and the pressure of the gas mixture is increased prior to contact with the withdrawn aqueous menstruum. Any unreacted hydrogen, carbon monoxide and carbon dioxide in the withdrawn substrate depleted gas is thus returned to the bioreactor assembly for bioconversion, thereby increasing conversion efficiency of the process. In some instances, the contact in step (f) between the withdrawn substrate depleted gas and withdrawn aqueous menstruum is under conditions where bioconversion of unreacted hydrogen and carbon dioxide and bioconversion of carbon monoxide, if present, can occur to produce additional oxygenated organic compound. This oxygenated organic compound will reside in the aqueous menstruum that is passed to product recovery unit operations.

In another independent aspect of this invention, recycling of substrate depleted gas from the bioreactor assembly is facilitated by its combination with at least a portion of the substrate gas which requires compression for introduction into the bioreactor assembly. The substrate depleted gas is generally rich in hydrogen, e.g., often hydrogen comprises between about 10 to 90, and more frequently between about 40 or 50 and 85, mole percent of the substrate depleted gas. Due to the low density of the substrate depleted gas in comparison to an exogenous carbon dioxide gas, which typically contains at least 20 volume percent carbon dioxide, the size of a compressor and energy requirements to compress the combination of gases are only slightly larger than those required to compress the exogenous carbon dioxide-containing gas alone. In its broad aspects, the processes of this aspect of the invention for the anaerobic bioconversion of a gas substrate comprising hydrogen and carbon dioxide, and optionally carbon monoxide, in an aqueous menstruum containing microorganisms suitable for converting said substrate to at least one oxygenated organic compound, comprise:
a. continuously introducing hydrogen-containing gas into a bioreactor assembly containing said aqueous menstruum as a portion of the gas substrate;
b. continuously introducing into said bioreactor assembly an exogenous carbon dioxide-containing gas, which introduction may be by one or more of direct introduction into said bioreactor assembly and admixture with at least a portion of the hydrogen-containing gas, said carbon dioxide-containing gas comprising a portion of the gas substrate;
c. maintaining contact between the aqueous menstruum and gas substrate under bioconversion conditions sufficient to bioconvert gas substrate to an oxygenated organic compound to provide a product-containing menstruum and a substrate depleted gas phase;
d. continuously withdrawing substrate depleted gas phase from said aqueous menstruum;
e. continuously or intermittently withdrawing a portion of said product-containing menstruum, said withdrawal being sufficient to maintain the oxygenated organic compound in said aqueous menstruum below a concentration that unduly adversely affects the microorganisms;
f. recovering said oxygenated organic compound from the withdrawn product-containing menstruum of step (e);
g. admixing a portion of the withdrawn substrate depleted gas with exogenous carbon dioxide gas to form a gas mixture; and
h. compressing said gas mixture to a pressure sufficient for introduction into the bioreactor assembly of step (b).

In some preferred embodiments, the compression of step (h) provides the gas mixture at a pressure suitable to introduction into the hydrogen-containing gas being supplied to the bioreactor assembly in accordance with step (a). Often the mixture is compressed to a pressure of between about 150 and 2000 kPa absolute, say, between about 150 and 1500 kPa absolute.

### BRIEF DESCRIPTION OF THE DRAWING

FIG. 1 is a schematic depiction of an apparatus useful in the practice of processes in accordance with this invention.

### DETAILED DISCUSSION

All patents, published patent applications and articles referenced herein are hereby incorporated by reference in their entirety.

### Definitions

As used herein, the following terms have the meanings set forth below unless otherwise stated or clear from the context of their use.

The use of the terms “a” and “an” is intended to include one or more of the element described.

The abbreviation ppm means parts per million. Unless otherwise stated or clear from the context, ppm is on a mole basis (ppm (mole)).

Alcohol means one or more alkanols containing two to six carbon atoms. In some instances the alcohol is a mixture of alkanols produced by the microorganisms contained in the aqueous menstruum.

Anaerobically derived gas means biogas produced by the anaerobic digestion or fermentation of organic matter in the absence of oxygen and primarily contains methane and carbon dioxide.

Aqueous menstruum means a liquid water phase which may contain dissolved compounds including, but not limited to hydrogen, carbon monoxide, and carbon dioxide. An aqueous menstruum can contain, but does not necessarily include the presence of, microorganisms for the bioconversion.

Biogas means a gas produced from a renewable source of carbon and preferably containing at least about 20 mole percent carbon dioxide.

Biomass means biological material living or recently living plants and animals and contains at least hydrogen, oxygen and carbon. Biomass typically also contains nitrogen, phosphorus, sulfur, sodium and potassium. The chemical composition of biomass can vary from source to source and even within a source. Sources of biomass include, but are not limited to, harvested plants such as wood, grass clippings and yard waste, switchgrass, corn (including corn stover), hemp, sorghum, sugarcane (including bagas), and the like; and waste such as garbage and municipal waste. Biomass does not include fossil fuels such as coal, natural gas, and petroleum.

A bioreactor assembly is an assembly of one or more vessels suitable to contain aqueous menstruum and microorganisms for the bioconversion and can contain associated equipment such as injectors, recycle loops, agitators, and the like.

Exogenous, oxygen-containing carbon dioxide gas is a carbon dioxide-containing gas that is sourced externally of the source of hydrogen-containing gas and the process for bioconverting substrate gas and recovering alcohol.

The term Component Composition means the composition of a gas where both water and nitrogen have been excluded from the calculation of the concentration of the components. As used herein, unless otherwise stated, compositions of gases are on an anhydrous basis and exclude the presence of nitrogen.

Deep tank bioreactor is a bioreactor having a depth of at least about 10 meters and can be operated to provide a substantial non-uniform substrate composition over the depth of the aqueous menstruum contained in the bioreactor. A commercial scale bioreactor has a capacity for aqueous menstruum of at least 1 million, and more preferably at least about 5, say, about 5 to 25 million, liters.

Coke oven gas has a typical composition of between about 1 and 5 volume percent carbon dioxide, about 3 and 10 volume percent carbon monoxide, about 20 and 40 volume percent methane, about 40 and 60 volume percent hydrogen with the balance being primarily nitrogen.

Effective Oxidation Reduction Potential is the oxidation reduction potential of the aqueous menstruum measured in millivolts. Where the aqueous menstruum is not substantially uniform in composition, then the determination of the Effective Oxidation Reduction Potential is determined from aqueous menstruum immediately prior to contact with exogenous, oxygen-containing carbon dioxide gas.

Electron to carbon ratio (e⁻/C) is calculated as the quotient of the quantity of two times the sum of the concentrations of carbon monoxide and hydrogen divided by quantity of the sum of the concentrations of carbon monoxide and carbon dioxide:

e⁻/C=2([CO]+[H₂])/([CO]+[CO₂]).

Intermittently means from time to time and may be at regular or irregular time intervals.

Landfill gas has the typical composition of between about 35 and 60 volume percent carbon dioxide and about 35 and 60 volume percent methane with small amounts of carbon monoxide, hydrogen, hydrogen sulfide, oxygen, and nitrogen.

Natural gas means a combustible mixture of gaseous hydrocarbons from sedimentary rocks usually containing over 75% methane with minor amounts of 2-4 carbon alkanes.

A concentration of oxygenated organic compound below that which unduly adversely affects the rate of growth of the culture of microorganisms will depend upon the type of microorganism and the oxygenated organic compound. An unduly adverse effect on the growth rate means that a significant, usually at least a 20 percent, decrease in the growth rate of the microorganisms is observed in comparison to the growth rate observed in an aqueous menstruum having about 10 grams per liter alcohol therein, all other parameters being substantially the same.

Oxygen, free oxygen and molecular oxygen mean the same and is represented by the chemical formula O₂.

Oxygenated organic compound means one or more organic compounds containing two to six carbon atoms selected from the group of aliphatic carboxylic acids and salts, alkanols and alkoxide salts, and aldehydes. Often oxygenated organic compound is a mixture of organic compounds produced by the microorganisms contained in the aqueous menstruum.

Substantial uniformity in liquid phase means that composition of the liquid phase is substantially the same throughout a bioreactor. Usually the concentration of the alcohol is within about 0.2 mole percentage points in a uniform liquid phase.

Substantial non-uniformity in the gas phase means that the concentration (both in the gas bubbles and dissolved) of at least one component provided by the gas substrate changes by at least 50 percent between the point of entry of the gas into a bioreactor and the point that the gas emerges from the aqueous menstruum.

Substrate gas is gas containing at least one of hydrogen, carbon dioxide and carbon monoxide. Where the substrate gas contains other components (other than water vapor), the substrate gas includes such other components. Total substrate gas is the fresh substrate gas introduced into the aqueous menstruum of a bioreactor assembly and thus does not include, e.g., recycled substrate depleted gas.

Syngas means a gas containing at least one of (i) hydrogen and carbon dioxide and (ii) carbon monoxide.

Renewable sources or renewable carbon means a source of carbon that can be replaced in less than a millennium and in most cases in several years or less. Nonrenewable gas stream means a gas derived from natural resources that takes at least a geologic age to replace once depleted and for this invention refers primarily to natural gas or methane stream derived fossil fuels.

### Overview

The processes of this invention bioconvert hydrogen-containing substrate gases to oxygenated organic compound using carbon dioxide co-substrate, at least a portion of which is exogenously supplied, using anaerobic fermentation wherein the concentration of any oxygen contained in the exogenous carbon dioxide-containing gas is reduced by contact with aqueous menstruum being passed to an oxygenated organic compound recovery unit operation.

### Syngas and Carbon Dioxide Sourcing

The syngas provides the hydrogen and, optionally, carbon monoxide, to the bioreactor assembly. The processes of this invention are particularly useful where the addition of carbon dioxide to the syngas provides useful substrate to the microorganisms for bioconversion to oxygenated organic compound. In some instances, the electron to carbon ratio of the syngas prior to its combination with the exogenous carbon dioxide is at least about 2:1, and is often at least about 4:1, and may be essentially pure hydrogen.

The source of the syngas is not critical to the broad aspects of this invention. Any suitable hydrogen-containing gas can be used as the synthesis gas such as coke oven gas or hydrogen-containing gas from the gasification, partial oxidation, and reforming (autothermal and steam) of biomass or fossil carbonaceous materials. Gasification and partial oxidation processes are disclosed in copending U.S. patent application Ser. No. 13/304,902, filed on Nov. 28, 2011, hereby incorporated by reference in its entirety. Rice, et al, in “Autothermal Reforming of Natural Gas to Synthesis Gas”, Reference: KBR Paper #2031, Sandia National Laboratories, April 2007, discuss autothermal reforming and conditions. Steam reforming is a widely practiced commercial unit operation. See Logdberg, et al., “Natural Gas Conversion”, Haldor Topsoe publication (undated). Reforming in the presence of carbon dioxide is known as carbon dioxide reforming with the partial pressure of carbon dioxide causing a shift in the product distribution of the reforming. See, for instance, Madsen, et al, “Industrial Aspects of CO₂-reforming”, Paper No. 28f, presented at the AIChE Spring Meeting, Houston, Tex., March 1997.

Steam reforming is generally preferred due to the high hydrogen concentration of the produced syngas and the relative absence of contaminants that must be removed to prevent deleterious effects on the microorganisms for the anaerobic bioconversion to oxygenated organic compound. Additionally, steam reforming, being non-oxidative, provides a syngas that is relatively free of nitrogen which would be present in the syngas produced by a partial oxidation or autothermal reforming process using air or enriched air as the oxygen source.

Since the unit operations to make the syngas can vary widely, it is understood that the compositions of the syngas may similarly vary widely including the presence of components other than hydrogen, carbon monoxide and carbon dioxide, which components may be inert such as nitrogen and methane or components that may have to be removed due to potential adverse effects on the microorganisms such as hydrogen cyanide. Processes for removing adverse components include those disclosed in U.S. patent application Ser. No. 13/304,902, filed on Nov. 28, 2011; Ser. No. 13/440,953, filed on Apr. 5, 2012; and Ser. No. 13/525,079, filed on Jun. 15, 2012; and U.S. Pat. No. 7,927,513 filed on Oct. 27, 2009 and U.S. Pat. No. 8,303,849, filed on Nov. 9, 2010. Also, the relative ratios among hydrogen, carbon monoxide and carbon dioxide may vary widely.

The exogenous carbon dioxide-containing gas for use in the processes of this invention can be obtained from any suitable source. Sources of the exogenous carbon dioxide-containing gas can be from nonrenewable sources or directly or indirectly from renewable sources. One convenient source of carbon dioxide is from the fermentation of carbohydrates to produce ethanol or other alkanols, diols and carboxylic acids or esters. The carbon dioxide may also be from an effluent gas from the bioconversion process. Another source of carbon dioxide-containing gas that can be used to adjust the composition of a syngas having a high hydrogen content is biogas such as from anaerobic digestion processes and from landfills. Process gases and gases from combustions that contain a high concentration of carbon dioxide may find utility however, such gases typically contain impurities that can adversely affect microorganisms. The processes of this invention inherently have the ability to remove at least a portion of components such as hydrogen cyanide, unsaturated aliphatic hydrocarbons, particulates and volatile metal compounds.

The ratio of syngas to exogenous carbon dioxide-containing gas can vary over a wide range. The ratio will be selected, among other things, on the sought electron to carbon ratio in the gas feed to the bioreactor assembly, the composition of the exogenous carbon dioxide-containing gas and the availability of such carbon dioxide gas. The carbon dioxide gas, after removal of oxygen, can be directly introduced into the bioreactor assembly without prior combination with the syngas or it can be combined with at least a portion of the syngas prior to its introduction into the bioreactor assembly. In either event, the relative amounts of the syngas and carbon dioxide gas supplied to the bioreactor assembly should provide an electron to carbon ratio of at least about 1:1, say, between about 2:1 to 6.8:1. The optimum electron to carbon ratio will depend upon the type of oxygenated organic compound sought. With alcohols, the optimum electron to carbon ratio is preferably in the range of about 5.2:1 or 5.7:1 to 6.4:1.

The exogenous carbon dioxide-containing gas used in the processes of this invention contains oxygen, e.g., at least about 100 parts per million by volume. The oxygen content of the exogenous carbon dioxide-containing gas will depend upon its source and handling. Oxygen is a minor portion of the exogenous carbon dioxide-containing gas, often less than about 5 volume percent, and preferably less than about 2 volume percent, say between about 500 or 1000 parts per million by volume to 1 volume percent. Most often the exogenous carbon dioxide-containing gas contains at least about 20 volume percent carbon dioxide. In situations where the exogenous carbon dioxide-containing gas contains less than about 50 volume percent carbon dioxide, it is preferred that at least one of hydrogen and carbon monoxide be present. Especially where the exogenous carbon dioxide-containing gas is sourced from carbohydrate fermentation operations, the gas (on a dry basis) contains at least about 90, say, 95 to virtually 100, volume percent carbon dioxide.

Removing Oxygen from the Exogenous Carbon Dioxide-Containing Gas

Oxygen is removed from the exogenous carbon dioxide-containing gas by contact with the aqueous menstruum which has been withdrawn from the bioreactor assembly for the recovery of the oxygenated organic compound. This aqueous menstruum from the anaerobic fermentation possesses a low oxidation reduction potential enabling a reduction of the free oxygen concentration in the exogenous carbon dioxide-containing gas. Preferably the aqueous menstruum also contains microorganisms, and preferably is an aliquot portion of the aqueous menstruum in the bioreactor assembly. Free oxygen can be toxic to the microorganisms used for anaerobic fermentations. While not wishing to be limited by theory, it is believed that these microorganisms can also serve to scavenge free oxygen as well as add to lowering the oxidation reduction potential of the aqueous medium. As this portion of the aqueous menstruum will be subjected to unit operations for recovery of the oxygenated organic compound, loss of the microorganisms will occur in any event.

The aqueous menstruum contacting the exogenous carbon dioxide-containing gas has an Effective Oxidation Reduction Potential of less than about −150, preferably less than about −250, and sometimes less than about −400 or −500, millivolts. As a general rule, lower Effective Oxidation Reduction Potentials are desirable. As discussed below, a supplemental reducing component may also be introduced to increase the capacity of the aqueous menstruum to lower the oxygen concentration of the aqueous menstruum. In some instances, the supplemental reducing component further decreases the oxidation reduction potential.

The contact between the exogenous carbon dioxide-containing gas and the aqueous menstruum can be provided by any suitable equipment capable of achieving gas-liquid contact including, but not limited to, those in which the gas is passed through the aqueous menstruum and those in which the gas is the continuous phase. Preferably a high contact surface area between the phases exists to enhance the mass transfer and reaction. Suitable apparatus include, but are not limited to, trickle bed contactors, spray absorbers and absorber columns, which can, if desired, contain baffles or packing to enhance contact. Preferably the contact between the aqueous menstruum and exogenous carbon dioxide-containing gas is countercurrent.

The temperature of the contacting may fall within a broad range. Often the aqueous menstruum is at a temperature close to that in the bioreactor assembly, which is favorable to the microorganisms continuing their metabolic activities. Especially where the exogenous carbon dioxide-containing gas contains hydrogen and/or carbon monoxide, the contacting may also enable additional oxygenated organic compound to be produced and sent to product recovery with the aqueous menstruum. Typically, the temperature of the contact is within the range of about 15° C. to 60° C., preferably between about 25° C. and 40° C.

The pressure of the exogenous carbon dioxide-containing gas to be contacted with the aqueous menstruum should at least be sufficient to enable the contacting and, preferably, allow it admixture with the hydrogen-containing gas or introduction into the bioreactor assembly, as the case may be. Often the pressure is at least about 110 kPa absolute, say, between about 150 and 2000, preferably 150 and 1500, kPa absolute.

The duration of the contact will depend upon the sought reduction of oxygen in the carbon dioxide gas, the nature of the equipment and conditions used for the contacting between the carbon dioxide gas and aqueous menstruum, and the oxidation reduction potential of the aqueous menstruum. In some instances, the micronutrients for the fermentation contain metals such as copper and cobalt that provide a catalytic effect and enhance the rate of the oxygen reduction. Typically the contact is sufficient to reduce the free oxygen concentration in the carbon dioxide gas to less than about 500, preferably less than about 250, and in some instances less than about 100, parts per million by volume, and often, based upon the initial concentration, the oxygen concentration is reduced by at least about 50, and more preferably at least by about 90, percent.

The degree of oxygen reduction will depend upon operating choices. Oxygen is an inhibitor and toxin to the anaerobic microorganisms. The operator may elect to allow some oxygen to pass to the bioreactor assembly. Although it will react in the bioreactor assembly and result in a lower conversion of gas substrate to oxygenated organic compound and can result in the inhibition or death of microorganisms in the bioreactor assembly, such loses may be tolerable. Usually, it is preferred to maintain the free oxygen concentration in the total substrate gas passed to the bioreactor assembly to less than about 100, more preferably less than about 30, parts per million by volume. Accordingly, if the exogenous carbon dioxide to be supplied is relatively small in comparison to the hydrogen-containing gas, higher concentrations of free oxygen in the carbon dioxide gas can be tolerated while still obtaining the sought overall rate of oxygen introduction into the bioreactor assembly.

The aqueous menstruum to contact the exogenous carbon dioxide-containing gas can be supplemented with reducing components to react with oxygen. The components can be exogenous to the process, such as bisulfite anion or, preferably, can be at least a portion of the substrate depleted gas from the bioreactor assembly which contains unreacted hydrogen and, if initially present, carbon monoxide. The amount of the supplemental reducing component, if required, provided to the aqueous menstruum is typically at least sufficient to enable the sought reduction of oxygen concentration in the carbon dioxide gas. The supplemental reducing components can thus conveniently be used to handle fluctuations in oxygen concentration in the exogenous carbon dioxide-containing gas. These fluctuations can occur as the source of the carbon dioxide changes and can occur even while using the same source. For instance, the oxygen concentration in a exogenous carbon dioxide-containing gas from a fermentation operation can vary as air is purged from bioreactors at start-up and due to leaks in piping and equipment. Similarly, biogas compositions are subject to change due to the process and equipment used for its generation. Further, the ability to use a supplemental reducing component enables the design of the contacting unit operation to be sized based upon a given concentration of oxygen in the exogenous carbon dioxide-containing gas and degree of removal, and use the supplemental reducing component to accommodate periods of time where the concentration of oxygen in the exogenous carbon dioxide-containing gas is greater or the volume of gas to be contacted with the aqueous medium is greater. Hence, in one preferred aspect of the processes of this invention, the rate of supply of supplemental reducing component is adjusted to maintain the carbon dioxide gas from the contacting with the aqueous menstruum below a predetermined level.

The addition of supplemental reducing components to the entails additional costs. However, since the primary source of reducing components is in the aqueous menstruum, the costs for reducing the oxygen concentration in the exogenous carbon dioxide-containing gas can be acceptable for a commercial-scale facility.

The use of the substrate depleted gas to supply supplemental reducing component to the aqueous menstruum for contact with the exogenous carbon dioxide-containing gas provides several advantages. First, at least some of the unreacted hydrogen and carbon monoxide, if present, is recycled to the bioreactor assembly thereby enhancing the bioconversion efficiency of the syngas. Second, where the aqueous menstruum contains viable microorganisms, at least a portion of the hydrogen and carbon monoxide, if present, can be bioconverted during the contact between the carbon dioxide gas and the aqueous menstruum. Third, as the pressure of the carbon dioxide gas needs to be sufficient to be contacted with the aqueous menstruum and to enter the bioreactor assembly, the compression unit operation can serve to also increase the pressure of a mixture of the substrate depleted gas and carbon dioxide gas thereby reducing capital costs for recycling substrate depleted gas to the bioreactor assembly. During periods of peak oxygen concentration in the exogenous carbon dioxide-containing gas, substantially all of the substrate depleted gas can be used. However, to maintain steady state conditions under normal operation, at least a portion of the substrate depleted gas needs to be purged, e.g., to prevent the build-up of inert gases such as nitrogen and methane. Hence, the portion of the substrate depleted gas directed to contact with the exogenous carbon dioxide-containing gas and aqueous menstruum at any given time can range from none to substantially all, but for extended durations of time, the portion of the substrate depleted gas used enables steady state operations to be maintained. Where a portion of the substrate depleted gas is used, the portion is preferably an aliquot portion.

In some embodiments of the processes of this invention, at least a portion of the substrate depleted gas is combined with the exogenous carbon dioxide-containing gas during steady state operations for recycle to the bioreactor. In these embodiments, the molar ratio of the substrate depleted gas and exogenous carbon dioxide-containing gas can vary widely and will be dependent, among other things, the portion of the substrate depleted gas sought to be recycled; the concentration of inert gases in the total substrate feed to avoid undue reduction of the mole fraction of hydrogen, and if present, carbon monoxide, to avoid undue reduction in the driving force for mass transfer into the aqueous phase; the conversion efficiency per pass in the bioreactor assembly and the relative amount of exogenous carbon dioxide-containing gas to provide the sought electron to carbon ratio. In some instances, the mole ratio of substrate depleted gas to exogenous carbon dioxide-containing gas is between about 5:1 to 1:5, and frequently between about 3:1 and 1:3.

### Oxygenated Organic Compound, Microorganisms and Fermentation Conditions

The oxygenated organic compounds produced in the processes of this invention will depend upon the microorganism used for the fermentation and the conditions of the fermentation. One or more microorganisms may be used in the aqueous menstruum to produce the sought oxygenated organic compound. Bioconversions of CO and H₂/CO₂ to acetic acid, propanol, butanol, butyric acid, ethanol and other products are well known. For example, in a recent book concise descriptions of biochemical pathways and energetics of such bioconversions have been summarized by Das, A. and L. G. Ljungdahl, Electron Transport System in Acetogens and by Drake, H. L. and K. Kusel, Diverse Physiologic Potential of Acetogens, appearing respectively as Chapters 14 and 13 of Biochemistry and Physiology of Anaerobic Bacteria, L. G. Ljungdahl eds, Springer (2003). Any suitable microorganisms that have the ability to convert the syngas components, CO, H₂, CO₂, individually or in combination with each other or with other components that are typically present in syngas may be utilized. Suitable microorganisms and/or growth conditions may include those disclosed in U.S. patent application Ser. No. 11/441,392, filed May 25, 2006, entitled “Indirect Or Direct Fermentation of Biomass to Fuel Alcohol,” (U.S. Published Patent Application No. 2007/0275447) which discloses a biologically pure culture of the microorganism Clostridium carboxidivorans having all of the identifying characteristics of ATCC no. BAA-624; U.S. Pat. No. 7,704,723 entitled “Isolation and Characterization of Novel Clostridial Species,” which discloses a biologically pure culture of the microorganism Clostridium ragsdalei having all of the identifying characteristics of ATCC No. BAA-622; both of which are incorporated herein by reference in their entirety. Clostridium carboxidivorans may be used, for example, to ferment syngas to ethanol and/or n-butanol. Clostridium ragsdalei may be used, for example, to ferment syngas to ethanol.

Suitable microorganisms and growth conditions include the anaerobic bacteria Butyribacterium methylotrophicum, having the identifying characteristics of ATCC 33266 which can be adapted to CO and used and this will enable the production of n-butanol as well as butyric acid as taught in the references: “Evidence for Production of n-Butanol from Carbon Monoxide by Butyribacterium methylotrophicum,” Journal of Fermentation and Bioengineering, vol. 72, 1991, p. 58-60; “Production of butanol and ethanol from synthesis gas via fermentation,” FUEL, vol. 70, May 1991, p. 615-619. Other suitable microorganisms include: Clostridium Ljungdahlii, with strains having the identifying characteristics of ATCC 49587 (U.S. Pat. No. 5,173,429) and ATCC 55988 and 55989 (U.S. Pat. No. 6,136,577) that will enable the production of ethanol as well as acetic acid; Clostridium autoethanogemum sp. nov., an anaerobic bacterium that produces ethanol from carbon monoxide. Jamal Abrini, Henry Naveau, Edomond-Jacques Nyns, Arch Microbiol., 1994, 345-351; Archives of Microbiology 1994, 161: 345-351; and Clostridium Coskatii having the identifying characteristics of ATCC No. PTA-10522 filed as U.S. Ser. No. 12/272,320 on Mar. 19, 2010.

Suitable microorganisms for bioconversion of syngas to oxygenated organic compounds generally live and grow under anaerobic conditions, meaning that dissolved oxygen is essentially absent from the aqueous menstruum. Adjuvants to the aqueous broth may comprise buffering agents, trace metals, vitamins, salts etc. Adjustments in the broth may induce different conditions at different times such as growth and non-growth conditions which will affect the productivity of the microorganisms. U.S. Pat. No. 7,704,723, hereby incorporated by reference in its entirety, discloses the conditions and contents of suitable aqueous broth for bioconversion CO and H₂/CO₂ using anaerobic microorganisms.

The bioreactor assembly may comprise one or more bioreactors which may be, with respect to gas flow, in parallel or in series flow. Representative bioreactor assemblies are bubble column bioreactors, stirred tank bioreactors, bioreactors having gas-lift riser section or sections, tubular bioreactors and membrane bioreactors. Deep tank bioreactors are often preferred due to economic considerations. Often deep tank bioreactors have a substantially uniform aqueous phase composition and a substantially non-uniform substrate concentration.

Anaerobic fermentation conditions include a suitable temperature, say, between 25° and 60° C., frequently in the range of about 30° to 40° C. The conditions of fermentation, including the density of microorganisms, aqueous broth composition, and syngas residence time, are preferably sufficient to achieve the sought conversion efficiency of hydrogen and carbon monoxide and will vary depending upon the design of the bioreactor assembly and its operation. The pressure may be subatmospheric, atmospheric or super atmospheric, and is usually in the range of from about 90 to 1500 KPa absolute and in some instances higher pressures may be desirable for biofilm fermentation reactors. As most bioreactor assembly designs, especially for commercial scale operations, provide for a significant height of aqueous menstruum for the fermentation, the pressure will vary within a bioreactor based upon the static head.

The fermentation conditions are preferably sufficient to effect at least about 40 or 50 percent conversion of the hydrogen in gas feed. For commercial operations, the fermentation operation preferably provides a total molar conversion of hydrogen and carbon monoxide in the net gas feed in the range of about 85 to 95 percent. Due to the low solubilities of carbon monoxide and hydrogen in the aqueous phase, achieving these high conversions may require a bioreactor assembly using one or more of multiple fermentation reactors and recycling off gas from a reactor. Typically in the case of multiple fermentation reactors it is only necessary to apply the processes of the invention to the first fermentation reactor.

The rate of supply of the gas substrate under steady state conditions to a fermentation reactor is such that the rate of transfer of carbon monoxide and hydrogen to the liquid phase matches the rate at which carbon monoxide and hydrogen are bioconverted. Hence, the dissolved concentration of carbon monoxide and hydrogen in the aqueous menstruum remains constant, i.e., does not build-up. The rate at which carbon monoxide and hydrogen can be consumed will be affected by the nature of the microorganism, the concentration of the microorganism in the aqueous menstruum and the fermentation conditions. As the rate of transfer of carbon monoxide and hydrogen to the aqueous menstruum is a parameter for operation, conditions affecting the rate of transfer, such as interfacial surface area between the gas and liquid phases and driving forces, are important.

### Substrate Depleted Gas Phase

The depleted gas phase egressing from the aqueous menstruum will contain a small fraction of the hydrogen and carbon oxides introduced into the bioreactor assembly as the substrate gas. Inerts such as nitrogen and primarily methane will comprise a significant portion of the depleted gas phase. The depleted gas phase may also contain sulfur-containing compounds, alcohol and the like volatilized from the aqueous menstruum.

### Product Recovery

The bioreactor assembly may have added from time to time or continuously one or more streams of water, nutrients or adjuvants, and microorganisms to maintain steady state operation. A portion of the aqueous menstruum is withdrawn from time to time or continuously from the bioreactor assembly for product recovery, at least a portion of which is used in the contact with exogenous carbon dioxide-containing gas to reduce its oxygen concentration. Product recovery can consist of known equipment arrangements for removal of residual cell material, separation and recovery of liquid products from the fermentation liquid, return of recovered fermentation liquid and purging of waste streams and materials. Suitable equipment arrangements can include filters, centrifuges, cyclones, distillation columns, membrane systems and other separation equipment. U.S. Pat. No. 8,211,679, herein incorporated by reference in its entirety, shows an arrangement for a product recovery bioreactor that recovers an ethanol product from a bioreactor.

### DRAWINGS

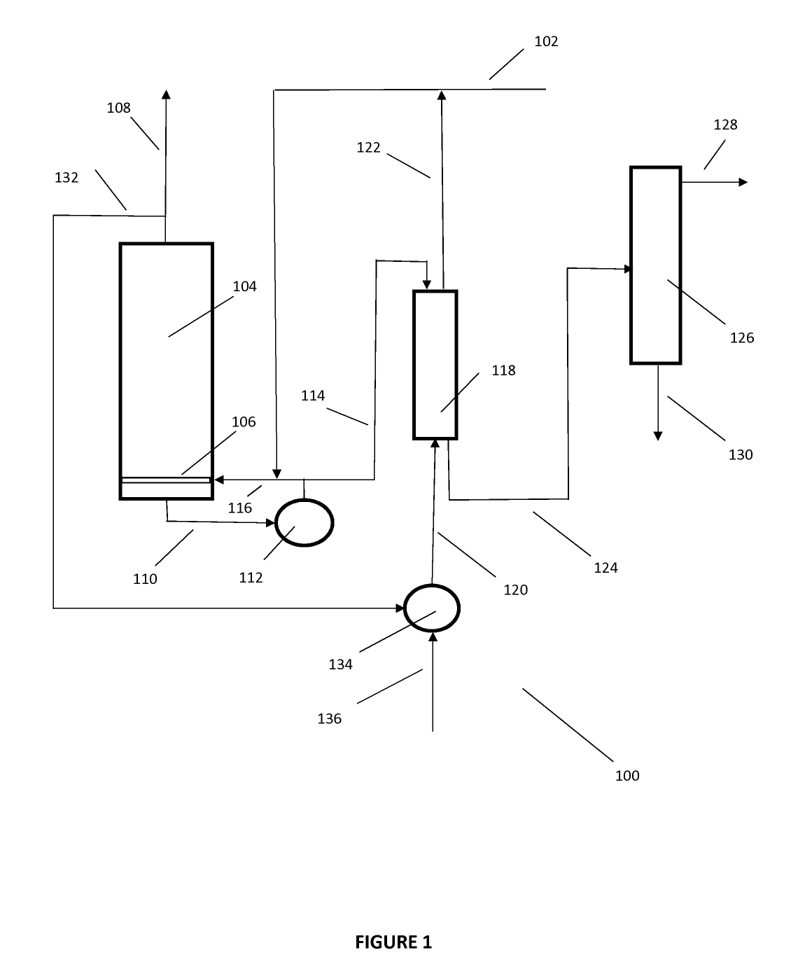

A general understanding of the invention and its application may be facilitated by reference to FIG. 1. The FIG. 1 is not in limitation of the broad aspects of the invention. FIG. 1 is a schematic depiction of an apparatus generally designated as 100 suitable for practicing the processes of this invention. FIG. 1 omits minor equipment such as pumps, compressors, valves, instruments and other devices the placement of which and operation thereof are well known to those practiced in chemical engineering. FIG. 1 also omits ancillary unit operations. The process and operation of FIG. 1 will be described in the context of the recovery and production of ethanol. The process is readily adaptable to making other alcohols such as i-butanol, n-butanol, and n-propanol and other oxygenated organic compounds.

A syngas produced by steam reforming of natural gas is provided via line 102 to bioreactor assembly 104. It should be recognized that other sources of hydrogen-containing gas can be used. Bioreactor assembly 104 is depicted as a deep tank bioreactor containing aqueous menstruum having microorganisms for bioconversion syngas to ethanol. Syngas is introduced at a lower section as small bubbles via distributor 106. Distributor 106 can be, for instance, a slot eductor using a recycling stream of aqueous menstruum provided by line 116 as the motive fluid. A substrate depleted gas exits at an upper portion of bioreactor assembly 104 via line 108.

A portion of the aqueous menstruum is withdrawn from bioreactor assembly 104 via line 110. Although depicted as withdrawing aqueous menstruum from a lower portion of bioreactor 104, the withdrawal may occur from a mid- or upper portion of bioreactor 104. The withdrawn aqueous menstruum is passed to pump 112 and an aliquot portion is returned to bioreactor 104 via line 116.

Another portion of the withdrawn aqueous menstruum is passed from pump 112 to absorber column 118 via line 114. Absorber column 118 provides for countercurrent contact between the liquid phase aqueous menstruum and an upwardly flowing carbon dioxide-containing gas phase supplied by line 120. An exogenous carbon dioxide-containing gas having a reduced oxygen concentration is withdrawn from an upper portion of absorber 118 via line 122 which directs the gas for mixing with the syngas supplied by line 102. Liquid phase (spent menstruum) is withdrawn from a lower portion of absorber 118 via line 124 where it is directed to distillation assembly 126. Distillation assembly 126 provides a lower boiling, ethanol-containing product stream which exits via line 128, and a bottoms stream which is predominantly water and contains solid debris from the microorganisms exits the distillation assembly via line 130.

As shown, a portion of the substrate depleted gas in line 108 is withdrawn via line 132 and passed to compressor 134. Also passed to compressor 134 via line 136 is the exogenous, oxygen-containing carbon dioxide-containing gas. The two gas streams are combined and compressor 134 directs the combined gases to absorber 118 via line 120.

It is claimed:

1. A continuous process for the anaerobic bioconversion of a gas substrate comprising hydrogen and carbon dioxide, and optionally carbon monoxide, in an aqueous menstruum containing microorganisms suitable for bioconverting said substrate to at least one oxygenated organic compound, the process comprising:
a. continuously introducing hydrogen-containing gas into a bioreactor assembly containing said aqueous menstruum as a portion of the gas substrate;
b. continuously introducing into said bioreactor assembly an exogenous carbon dioxide-containing gas, which introduction may be by one or more of direct introduction into said bioreactor assembly and admixture with at least a portion of the hydrogen-containing gas, said carbon dioxide-containing gas comprising a portion of the gas substrate;
c. maintaining contact between the aqueous menstruum and gas substrate under bioconversion conditions sufficient to bioconvert gas substrate to an oxygenated organic compound, said bioconversion conditions including an Effective Oxidation Reduction Potential of less than about −150 to provide a product-containing menstruum and a substrate depleted gas phase;
d. continuously withdrawing substrate depleted gas phase from said aqueous menstruum;
e. continuously or intermittently withdrawing a portion of said product-containing menstruum, said withdrawal being sufficient to maintain the oxygenated organic compound in said menstruum below a concentration that unduly adversely affects the microorganisms;
f. contacting at least a portion of the withdrawn product-containing menstruum with an exogenous, oxygen-containing, carbon dioxide gas under conditions sufficient to reduce the oxygen concentration of the carbon dioxide gas and to provide a spent menstruum;
g. passing at least a portion of the carbon dioxide gas having a reduced oxygen concentration of step (f) to the bioreactor assembly as at least a portion of the carbon dioxide-containing gas of step (b); and
h. recovering said oxygenated organic compound from the spent menstruum of step (f).

2. The process of claim 1 wherein the Effective Oxidation Reduction Potential of the aqueous menstruum in step (c) is less than about −250 millivolts.

3. The process of claim 1 wherein the Effective Oxidation Reduction Potential of the withdrawn product menstruum in step (f) is less than about −250 millivolts.

4. The process of claim 3 wherein the Effective Oxidation Reduction Potential of the withdrawn product menstruum in step (f) is less than about −400 millivolts.

5. The process of claim 1 wherein sufficient exogenous carbon dioxide is provided in step (b) to provide a total substrate gas having an electron to carbon ratio of between about 2:1 to 6.8:1.

6. The process of claim 1 wherein a supplemental reducing component is provided in step (f).

7. The process of claim 6 wherein the amount of supplemental reducing component provided in step (f) varies with the concentration of oxygen in the exogenous, oxygen-containing carbon dioxide gas.

8. The process of claim 6 wherein the supplemental reducing component comprises at least a portion of the substrate depleted gas of step (d).

9. The process of claim 1 wherein the hydrogen-containing gas is derived from steam reforming.

10. The process of claim 1 wherein the exogenous, oxygen-containing carbon dioxide gas is from the off gas of a carbohydrate fermentation.

11. The process of claim 1 wherein the oxygenated organic compound comprises alcohol.

12. The process of claim 11 wherein the alcohol comprises ethanol.

13. The process of claim 11 wherein the alcohol comprises butanol.

14. The process of claim 1 wherein the exogenous, oxygen-containing carbon dioxide gas contains at least about 1000 parts per million by volume free oxygen.

15. The process of claim 1 wherein the exogenous, oxygen-containing carbon dioxide gas contains less than about 2 volume percent free oxygen.

16. A process for the anaerobic bioconversion of a gas substrate comprising hydrogen and carbon dioxide, and optionally carbon monoxide, in an aqueous menstruum containing microorganisms suitable for converting said substrate to at least one of an oxygenated organic compound, the process comprising:
a. continuously introducing hydrogen-containing gas into a bioreactor assembly containing said aqueous menstruum as a portion of the gas substrate;
b. continuously introducing into said bioreactor assembly an exogenous carbon dioxide-containing gas, which introduction may be by one or more of direct introduction into said bioreactor assembly and admixture with at least a portion of the hydrogen-containing gas, said carbon dioxide-containing gas comprising a portion of the gas substrate;
c. maintaining contact between the aqueous menstruum and gas substrate under bioconversion conditions sufficient to bioconvert gas substrate to an oxygenated organic compound to provide a product-containing menstruum and a substrate depleted gas phase;
d. continuously withdrawing substrate depleted gas phase from said aqueous menstruum;
e. continuously or intermittently withdrawing a portion of said product-containing menstruum, said withdrawal being sufficient to maintain the oxygenated organic compound in said aqueous menstruum below a concentration that unduly adversely affects the microorganisms;
f. recovering said oxygenated organic compound from the withdrawn product-containing menstruum of step (e);
g. admixing a portion of the withdrawn substrate depleted gas with exogenous carbon dioxide gas to form a gas mixture; and
h. compressing said gas mixture to a pressure sufficient for introduction into the bioreactor assembly of step (b).

17. The process of claim 16 wherein the mixture is compressed to a pressure of between about 150 and 2000 kPa absolute.

18. The process of claim 16 wherein the mole ratio of substrate depleted gas to exogenous carbon dioxide-containing gas is between about 5:1 to 1:5.
